(19) United States
(12) Patent Application Publication
(10) Pub. No.: US 2013/0029877 A1
(43) Pub. Date: Jan. 31, 2013

(54) METHODS FOR OPTIMIZING OPTICAL MAPPING CONDITIONS

(71) Applicants: Colin William Dykes, Gaithersburg, MD (US); Trevor Wagner, Madison, WI (US); Adam Michael Briska, Madison, WI (US); Wenlong Jiang, Madison, WI (US)

(72) Inventors: Colin William Dykes, Gaithersburg, MD (US); Trevor Wagner, Madison, WI (US); Adam Michael Briska, Madison, WI (US); Wenlong Jiang, Madison, WI (US)

(73) Assignee: OPGEN, INC., Gaithersburg, MD (US)

(21) Appl. No.: 13/190,934

(22) Filed: Jul. 26, 2011

(51) Int. Cl. C40B 30/08; C12M 1/34; C40B 60/12

(52) U.S. Cl. 506/11; 506/39; 435/287.2

(57) ABSTRACT

The invention generally relates to methods and apparatuses for optimizing conditions for optical mapping. In certain embodiments, methods of the invention involve providing a substrate including a gradient of silanes in a first direction, introducing to the substrate, a gradient of enzyme activity in a second direction, contacting a plurality of enzymes and a plurality of nucleic acids to the substrate, and analyzing enzymatic activity and interaction of the nucleic acids with the substrate, thereby determining the optimal conditions for optical mapping of the nucleic acid.

### FIELD OF THE INVENTION

The invention generally relates to methods and apparatuses for optimizing conditions for optical mapping.

### BACKGROUND

Physical mapping of genomes, e.g., using restriction endonucleases to develop restriction maps, can provide accurate information about the nucleic acid sequences of various organisms. Restriction maps of, e.g., deoxyribonucleic acid (DNA), can be generated by optical mapping. Optical mapping can produce ordered restriction maps by using fluorescence microscopy to visualize restriction endonuclease cutting events on individual labeled DNA molecules.

In optical mapping, DNA is digested by a restriction enzyme on a glass surface. Many factors may influence digestion of the DNA, such as total amount of enzyme activity in which the DNA is subjected. Total enzyme activity is governed by may factors, such as amount of enzyme present for the digestion, specific activity of the enzyme (units of activity per microgram of protein), temperature at which digestion occurs, and amount of time the DNA is exposed to the enzyme.

Digestion is also affected by the manner in which the glass surface is treated to impart a net positive charge required to capture the target DNA by electrostatic interaction. A high positive charge density is associated with efficient capture and holding DNA molecules before digestion, and retention of small restriction fragments after digestion. However, a high positive charge also reduces digestion efficiency, because the DNA is held tightly to the positively charged surface and is less accessible for digestion. Conversely, a low charge density is associated with higher digestion efficiency (digest times are greatly reduced). However, DNA is not captured as efficiently and there is a higher rate of loss of small fragments from the surface.

Since there is a fine balance between good digestion and good DNA capture/retention, combinations of surfaces treated with different concentrations of silanes (to impart the required positive charge) and digested for different times are tested empirically to assess the best combination, which is both time consuming and expensive.

There is a need for more efficient and less expensive methods and apparatuses for optimizing conditions for optical mapping.

### SUMMARY

The invention generally relates to methods and apparatuses for optimizing conditions for optical mapping. Methods of the invention simultaneously analyze multiple parameters on a single preparation that effect enzyme activity and nucleic acid interaction with a substrate. Thus methods of the invention eliminate the need for multiple surface preparations and multiple assays, saving time and costs.

Aspects of the invention are accomplished by providing a substrate having a gradient of silanes in a first direction. The silanes produce a net charge on the substrate that is required for capture/retention of nucleic acids. The gradient of silanes allows for evaluation of nucleic acid interaction with the substrate, and allows for determination of an optimal concentration of silanes on the substrate.

A gradient of enzyme activity is then introduced in a second direction to the substrate. One of skill in the art will be aware of numerous parameters that can be used to measure enzymatic activity, any of which are suitable with methods and apparatuses of the present invention. Exemplary factors that effect enzyme activity include, temperature, interaction time between the enzyme and the nucleic acid, enzyme concentration, or external agents that modulate enzyme activity. A plurality of enzymes and a plurality of nucleic acids are then contacted to the substrate. Because the silane gradient and the enzyme activity gradient are in different dimensions, analysis of multiple parameters that effect enzyme activity and nucleic acid interaction with a substrate occur simultaneously. Further, a large number of different combinations of binding and activity are produced, ensuring that an optimal combination is generated on the substrate.

Based on the analysis of the multiple parameters, the optimal combination of binding and activity is determined, and this combination is used for subsequent restriction digests of nucleic acids from organisms. The restriction digests are then assembled into an optical map. Exemplary organisms include a microorganism, a bacterium, a virus, and a fungus. To facilitate observation of binding and activity, the nucleic acids may be labeled prior to introducing them to the substrate.

The first and second gradient may be either continuous or discontinuous. The substrate may be composed of any material that is compatible with optical mapping. An exemplary material is glass. Methods of the invention may be used to evaluate any enzymes. In particular embodiments, methods of the invention are used to evaluate restriction enzymes. Exemplary restriction enzymes include BglII, NcoI, XbaI, and BamHI. The enzymes spaced across the surface may be all the same enzyme. Alternatively, the plurality of enzymes spaced across the surface may be different enzymes.

Another aspect of the invention relates to methods and apparatuses for assessing activity of an enzyme for digestion of nucleic acids. Methods and apparatuses of the invention take advantage of the correlation between enzyme activity and temperature. Aspects of the invention are accomplished by applying a temperature gradient to a surface including a plurality of enzymes and a plurality of nucleic acids spaced across at least a portion of the surface. The temperature gradient produces a continuous range of enzyme activity across the surface, thus allowing for an assessment of enzyme activity across various temperatures on a single surface and in a single assay, eliminating the need for multiple surface preparations and multiple assays. Based on the assessment of enzyme activity, a range of temperatures at which the restriction enzyme will digest nucleic acid with high fidelity and with high efficiency is determined. In particular embodiments, an optimal temperature for enzymatic digestion of a nucleic acid is determined.

One of skill in the art will be aware of numerous parameters that can be used to measure enzymatic activity, any of which are suitable with methods and apparatuses of the present invention. Exemplary parameters include average fragment size of the digested nucleic acids, gap size, and digestion rate of an internal standard.

Another aspect of the invention provides an apparatus for assessing activity of a restriction enzyme including a heating/cooling device coupled to a substrate, in which the heating/cooling device generates a temperature gradient across at least a portion of the substrate. Any device that is capable of generating a temperature gradient can be used with the apparatuses of the invention. An exemplary heating/cooling device is a Peltier device. The apparatus can further include an imaging device.

### DETAILED DESCRIPTION

The invention generally relates to methods and apparatuses for optimizing conditions for optical mapping. An aspect of the invention provides methods for optimizing conditions for optical mapping of a nucleic acid including providing a substrate including a gradient of silanes in a first direction, introducing to the substrate, a gradient of enzyme activity in a second direction, contacting a plurality of enzymes and a plurality of nucleic acids to the substrate, and analyzing enzymatic activity and interaction of the nucleic acids with the substrate, thereby determining the optimal conditions for optical mapping of the nucleic acid.

The surface can be composed of any material that is suitable for optical mapping and is compatible with nucleic acids. Exemplary materials include polymers, ceramics, glass, or metals. In a preferred embodiment, the surface is glass, such as a microscope slide. Because a net negative charge is require to capture/retain nucleic acids, the surface includes silanes to impart a net negative charge to the surface. However, digestion of the nucleic acid by the enzyme is effected by the manner in which the glass surface is treated to impart the net positive charge required to capture the target DNA by electrostatic interaction. A high positive charge density is associated with efficient capture and holding DNA molecules before digestion, and retention of small restriction fragments after digestion. However, a high positive charge also reduces digestion efficiency, because the DNA is held tightly to the positively charged surface and is less accessible for digestion. Conversely, a low charge density is associated with higher digestion efficiency (digest times are greatly reduced). However, DNA is not captured as efficiently and there is a higher rate of loss of small fragments from the surface. Thus a gradient of silanes is applied to the substrate in a first direction, i.e., varying silane concentration across the substrate. This allows for evaluation of the effect of varying silane concentrations on nucleic acid capture/retention.

A gradient of enzyme activity is then introduced in a second direction. In particular embodiments, the second direction is substantially perpendicular to the gradient of silanes in a first direction. One of skill in the art will be aware of numerous parameters that can be used to measure enzymatic activity, any of which are suitable with methods and apparatuses of the present invention. Exemplary factors that affect enzyme activity include, temperature, interaction time between the enzyme and the nucleic acid, enzyme concentration, or external agents that modulate enzyme activity.

A temperature gradient may be generated by using a peltier heating/cooling device coupled with a reactive surface. Driving the peltier to produce a heating effect on one side and a cooling effect on the other will produce a strong temperature gradient across the substrate. This strong temperature gradient will in turn produce a continuous range of enzyme activity across the substrate, as enzyme activity is strongly correlated with temperature.

In certain embodiments, the gradient of enzyme activity may be generated by gradually flowing a solution containing the restriction endonuclease across the surface, at right angles to the direction of the silane gradient, such that different parts of the surface are in contact with the enzyme for different amounts of time.

In other embodiments, the gradient of enzyme activity may be created by exposing the surface to a gradient of solution containing different concentrations of restriction enzyme, or containing gradients of compounds that modulate restriction enzyme activity either positively or negatively.

Nucleic acids (e.g., deoxyribonucleic acid or ribonucleic acid) are applied to the substrate along with at least one enzyme. The nucleic acids and enzymes are applied such that they are spaced across at least a portion of the surface. In certain embodiments, the nucleic acids and enzymes are spaced across the entire surface. Methods for applying nucleic acids to a surface are well known to one of skill in the art. See for example U.S. Pat. No. 5,405,519, U.S. Pat. No. 5,599,664, U.S. Pat. No. 6,150,089, U.S. Pat. No. 6,147,198, U.S. Pat. No. 5,720,928, U.S. Pat. No. 6,174,671, U.S. Pat. No. 6,294,136, U.S. Pat. No. 6,340,567, U.S. Pat. No. 6,448,012, U.S. Pat. No. 6,509,158, U.S. Pat. No. 6,610,256, and U.S. Pat. No. 6,713,263, each of which is incorporated by reference herein.

Prior to application to the slide, the nucleic acids can be labeled, which can assist is measuring certain parameters of enzymatic activity. Labeling methods are known in the art and can include any known label. However, preferred labels are optically-detectable labels, such as 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron® Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; naphthalo cyanine, BOBO, POPO, YOYO, TOTO and JOJO.

Exemplary nucleic acids include deoxyribonucleic acid or ribonucleic acid. A wide size range of nucleic acid molecules, i.e., from about 300 bp to mammalian chromosome-size (that is greater than 1000 kb) can efficiently be applied onto the surfaces described herein. In a particular embodiment, methods of the invention are used to determine the optimal enzyme activity for a restriction endonuclease, e.g., BglII, NcoI, XbaI, and BamHI, to digest a nucleic acid. In certain embodiments, a single enzyme is assessed. In other embodiments, a plurality of different enzymes are assessed. Exemplary combinations of restriction enzymes include:

| | | |
|---|---|---|
| AflII | ApaLI | BglII |
| AflII | BglII | NcoI |
| ApaLI | BglII | NdeI |
| AflII | BglII | MluI |
| AflII | BglII | PacI |
| AflI | MluI | NdeI |
| BglII | NcoI | NdeI |
| AflII | ApaLI | MluI |
| ApaLI | BglII | NcoI |
| AflII | ApaLI | BamHI |
| BglII | EcoRI | NcoI |
| BglII | NdeI | PacI |
| BglII | Bsu36I | NcoI |
| ApaLI | BglII | XbaI |
| ApaLI | MluI | NdeI |
| ApaLI | BamHI | NdeI |
| BglII | NcoI | XbaI |
| BglII | MluI | NcoI |
| BglII | NcoI | PacI |
| MluI | NcoI | NdeI |
| BamHI | NcoI | NdeI |
| BglII | PacI | XbaI |
| MluI | NdeI | PacI |
| Bsu36I | MluI | NcoI |
| ApaLI | BglII | NheI |
| BamHI | NdeI | PacI |
| BamHI | Bsu36I | NcoI |
| BglII | NcoI | PvuII |
| BglII | NcoI | NheI |
| BglII | NheI | PacI |

After a period of incubation, the substrate is then imaged (e.g., using a fluorescent microscope). By having a silane gradient in a first direction and an enzyme activity gradient in a second direction, methods of the invention provide a substrate having regions that carry different concentrations of bound silanes and regions subjected to different amounts of restriction enzyme activity, and thus allow for particular regions of the surface to exhibit conditions that are optimal for capture/digestion/retention of a nucleic acid. Based on the analysis of the multiple parameters, the optimal combination of binding and activity is determined. The optimal binding and activity conditions can be used in any further work for which the conditions were assessed. In certain embodiments, the assessed conditions are then applied to generation of an optical map, which is discussed below.

Another aspect of the invention generally relates to methods and apparatuses for assessing activity of an enzyme for digestion of nucleic acids. The methods take advantage of the fact that each enzyme has an optimum temperature at which it most efficiently digests nucleic acid. A higher temperature generally results in an increase in enzymatic activity. As the temperature increases, molecular motion increases resulting in more molecular collisions. However, if the temperature rises above a certain point, the heat will denature the enzyme, resulting in the enzyme losing its three-dimensional functional shape by denaturing its hydrogen bonds and thus decreasing the activity of the enzyme. Further, if the temperature rises above a certain point, fidelity of the enzyme will be compromised, i.e., the enzyme will cleave sequences that are similar but not identical to their defined recognition sequence (star activity). In contrast, cold temperature decreases enzymatic activity by decreasing molecular motion.

These methods involve applying a temperature gradient to a solid surface including a plurality of enzymes and a plurality of nucleic acids, in which the enzymes and the nucleic acids are spaced across at least a portion of the solid surface, and analyzing fidelity and efficiency of the enzyme to digest the nucleic acid across the temperature gradient, thereby assessing activity of the enzyme.

The surface can be composed of any material that is suitable for optical mapping and is compatible with nucleic acids. Exemplary materials include polymers, ceramics, glass, or metals. In a preferred embodiment, the surface is glass, such as a microscope slide. Nucleic acids (e.g., deoxyribonucleic acid or ribonucleic acid) are applied to the substrate along with at least one enzyme. The nucleic acids and enzymes are applied such that they are spaced across at least a portion of the surface. Prior to application to the slide, the nucleic acids can be labeled, which can assist is measuring certain parameters of enzymatic activity. Labeling methods are known in the art and can include any known label.

The surface is coupled to a heating/cooling device that is capable of producing a temperature gradient across the surface. Any device that is capable of generating a temperature gradient can be coupled to the surface. An exemplary heating/cooling device is a Peltier device (commercially available from Custom Thermoelectric, Bishopville Md.). Peltier devices, also known as thermoelectric (TE) modules, are small solid-state devices that function as heat pumps. Generally, the device is formed by two ceramic plates with an array of small Bismuth Telluride cubes in between. Application of a DC current moves heat from one side of the device to the other, thus producing a temperature gradient in which a first side to which the device is connected is cooled and a second side to which the device is connected is heated. To increase the efficiency of the Peltier module, a thermal interface material can be placed between the Peltier module and the surface. Exemplary thermal interface materials include silicone-based greases (e.g., zinc oxide silicone), elastomeric pads, thermally conductive tapes, and thermally conductive adhesives.

The temperature gradient produces a continuous range of enzyme activity across the surface. The temperature gradient to be used will depend on the particular enzyme, and can be determined by one of skill in the art. The gradient can range from about 0° C. to about 150° C., or from about 0° C. to about 80° C., or from about 0° C. to about 60° C., or from about 0° C. to about 50° C., or from about 10° C. to about 150° C., or from about 10° C. to about 80° C., or from about 10° C. to about 60° C., or from about 10° C. to about 50° C., or from about 20° C. to about 100° C., or from about 20° C. to about 80° C., or from about 20° C. to about 60° C., or from about 20° C. to about 50° C., etc.

After a period of incubation, the surface is then imaged (e.g., using a fluorescent microscope) and at least one parameter indicative of enzyme activity is assessed. For example, the digestion rate across the surface can be measured by the average fragment size of the digested nucleic acids, gap size, or digestion rate of an internal standard. Various types of internal standards/references can be used during restriction mapping. One type of a standard is an internal fluorescence standard consisting of a reference DNA molecule of known sequence. Other measurable parameters of enzyme activity will be known to those of skill in the art. See for example Peterson et al. (Biochem J., 402(Pt 2):331-337, 2007). From these parameters, activity of the enzyme to digest nucleic acids is determined based upon the known temperature gradient.

The assessed activity of the enzyme can be used in any further work involving the particular enzymes for which the conditions were assessed. In certain embodiments, the assessed conditions for digestion of nucleic acids is then applied to generation of an optical map. Optical mapping is a single-molecule technique for production of ordered restriction maps from a single DNA molecule (Samad et al., Genome Res. 5:1-4, 1995).

Various methods can be used for controllable elongation of single nucleic acid molecules in optical mapping and/or sequencing. The methods can be gel-based, solid surface-based, and flow-based (see, e.g., U.S. Pat. No. 6,509,158). During some applications, individual fluorescently labeled DNA molecules are elongated in a flow of agarose between a coverslip and a microscope slide (in a first-generation method) or fixed onto polylysine-treated glass surfaces (in a second-generation method). Samad et al. supra. The added endonuclease cuts the DNA at specific points, and the fragments are imaged. Id. Restriction maps can be constructed based on the number of fragments resulting from the digest. Id. Generally, the final map is an average of fragment sizes derived from similar molecules. Id.

Optical mapping and related methods are described in U.S. Pat. No. 5,405,519, U.S. Pat. No. 5,599,664, U.S. Pat. No. 6,150,089, U.S. Pat. No. 6,147,198, U.S. Pat. No. 5,720,928, U.S. Pat. No. 6,174,671, U.S. Pat. No. 6,294,136, U.S. Pat. No. 6,340,567, U.S. Pat. No. 6,448,012, U.S. Pat. No. 6,509,158, U.S. Pat. No. 6,610,256, and U.S. Pat. No. 6,713,263. All the cited patents are incorporated by reference herein in their entireties.

Optical Maps are constructed as described in Reslewic et al., Appl Environ Microbiol. 2005 September; 71 (9):5511-22, incorporated by reference herein. Briefly, individual chromosomal fragments from test organisms are immobilized on derivatized glass by virtue of electrostatic interactions between the negatively-charged DNA and the positively-charged surface, digested with one or more restriction endonuclease, stained with an intercalating dye such as YOYO-1 (Invitrogen) and positioned onto an automated fluorescent microscope for image analysis. Since the chromosomal fragments are immobilized, the restriction fragments produced by digestion with the restriction endonuclease remain attached to the glass and can be visualized by fluorescence microscopy, after staining with the intercalating dye. The size of each restriction fragment in a chromosomal DNA molecule is measured using image analysis software and identical restriction fragment patterns in different molecules are used to assemble ordered restriction maps covering the entire chromosome.

Restriction mapping, e.g., optical mapping, can be used in a variety of applications. For example, the methods featured herein can be used to determine a property, e.g., physical and/or chemical property, e.g., size, length, restriction map, weight, mass, sequence, conformational or structural change, pKa change, distribution, viscosity, rates of relaxation of a labeled and/or non-labeled molecule, e.g., an amplicon (e.g., PCR product), of a portion of a genome (e.g., a chromosome), or of an entire genome.

The methods can also be used to identify various organisms, e.g., viruses and prions, and various microorganisms, e.g., bacteria, protists, and fungi, whose genetic information is stored as DNA or RNA by correlating the restriction map of a nucleic acid of an organism with a restriction map database. Such identification methods can be used in diagnosing a disease or disorder. Methods of identifying organisms by restriction mapping are described, e.g., in a U.S. patent application Ser. No. 12/120,586, filed on May 14, 2008, incorporated herein by reference.

The methods featured herein can also be used in other diagnostic applications, for example, imaging specific loci or genetic regions for individuals or populations to help identify specific diseases or disorders. Other uses of the methods will be apparent to those skilled in the art.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

1. A method for optimizing conditions for optical mapping of a nucleic acid, the method comprising:
providing a substrate comprising a gradient of silanes in a first direction;
introducing to the substrate, a gradient of enzyme activity in a second direction;
contacting a plurality of enzymes and a plurality of nucleic acids to the substrate; and
analyzing enzymatic activity and interaction of the nucleic acids with the substrate, thereby determining the optimal conditions for optical mapping of the nucleic acid.

2. The method according to claim 1, wherein the nucleic acids are optically labeled prior to the contacting step.

3. The method according to claim 1, wherein the gradient of silanes is continuous.

4. The method according to claim 1, wherein the gradient of silanes is discontinuous.

5. The method according to claim 1, wherein the gradient of enzyme activity is continuous.

6. The method according to claim 1, wherein the gradient of enzyme activity is discontinuous.

7. The method according to claim 1, wherein the enzymes are restriction enzymes.

8. The method according to claim 1, wherein the plurality of enzymes are the same enzymes.

9. The method according to claim 1, wherein the plurality of enzymes are different enzymes.

10. The method according to claim 1, wherein the gradient of enzyme activity is selected from the group consisting of: a temperature gradient; a time gradient; an enzyme concentration gradient; and a gradient of compounds that modulate enzyme activity.

11. The method according to claim 1, wherein the first direction and the second direction are substantially perpendicular to each other.

12. The method according to claim 1, wherein the substrate is glass.

13. The method according to claim 11, further comprising digesting nucleic acid from an organism with one or more of the enzymes under conditions determined from the analyzing step, and preparing an optical map of the restriction digests.

14. A method for assessing activity of a restriction enzyme, the method comprising:
applying a temperature gradient to a substrate comprising a plurality of enzymes and a plurality of nucleic acids, wherein the enzymes and the nucleic acids are spaced across at least a portion of the substrate; and
analyzing fidelity and efficiency of the enzyme to digest the nucleic acid across the temperature gradient, thereby assessing activity of the enzyme.

15. A method for determining an optimal temperature for enzymatic digestion of a nucleic acid, the method comprising:
applying a temperature gradient to a substrate comprising a plurality of enzymes and a plurality of nucleic acids, wherein the enzymes and the nucleic acids are spaced across at least a portion of the substrate; and
determining an optimal temperature for enzymatic digestion of the nucleic acid based upon enzymatic activity across the substrate.

16. A method for determining an optimal concentration of silanes on a substrate for optical mapping, the method comprising:
applying a plurality of enzymes and a plurality of nucleic acids spaced across at least a portion of a substrate, wherein the substrate comprises regions having different silane concentrations; and
analyzing enzymatic activity and interaction of the nucleic acids with the substrate, thereby determining the optimal concentration of silanes for optical mapping.

17. An apparatus for assessing activity of a restriction enzyme, the apparatus comprising: a heating/cooling device coupled to a substrate, wherein the heating/cooling device generates a temperature gradient across at least a portion of the substrate.

18. The apparatus according to claim 17, wherein the substrate is glass.

19. The apparatus according to claim 18, wherein the heating/cooling device is a Peltier device.

20. The apparatus according to claim 19, further comprising an imaging device.
